(19) United States
(12) Patent Application Publication
(10) Pub. No.: US 2010/0298386 A1
(43) Pub. Date: Nov. 25, 2010

(54) COMPOSITIONS AND METHODS FOR TREATING MASTITIS

(76) Inventor: Steven R. Burwell, San Juan, PR (US)

Correspondence Address: SUTHERLAND ASBILL & BRENNAN LLP, 999 PEACHTREE STREET, N.E., ATLANTA, GA, 30309, US

(21) Appl. No.: 12/681,666

(22) PCT Filed: Oct. 2, 2008
(86) PCT No.: PCT/US08/11393

§ 371 Date: Jul. 20, 2010

(30) Foreign Application Priority Data
Oct. 3, 2007 (US) 60997475

(51) Int. Cl. A61K 31/4425; A61P 31/00; A61K 31/14

(52) U.S. Cl. 514/358; 514/643

(57) ABSTRACT

The present disclosure relates to compositions that are effective in controlling or in preventing mastitis in an animal. The disclosed compositions comprise a biocidal system, comprising a primary biocide and a preservative component; a nonionic surfactant having an HLB of from about 10 to about (20); an emollient system comprising an extradermal penetrating agent and an emollient base; a thickening agent; and an aqueous based carrier.

### CROSS REFERENCE TO RELATED APPLICATIONS

This application claims the benefit of U.S. Provisional Application No. 60/997,475, filed Oct. 3, 2007. U.S. Provisional Application No. 60/997,475 is hereby incorporated herein by reference in its entirety.

### FIELD OF THE DISCLOSURE

The present disclosure relates to compositions that are effective in controlling or in preventing mastitis in an animal.

### BACKGROUND OF THE DISCLOSURE

Bovine mastitis is an inflammation of the udder. This condition, which is almost exclusively initiated by pathogenic microorganisms that have entered the teat canal after the milking process, occludes milk flow and production, and can permanently impair an animal's future ability to produce milk. The usual sources of harmful microorganisms include unsanitary milking equipment, the milker, other mastitic animals, an unsanitary stable/pen environment, and the animal's own elimination (defecation/urination) processes. It is estimated that each year hundreds of millions of dollars are lost to this disease in the United States alone. Estimates of total annual milk product lost in the United States due to mastitis range from 10 to 25 percent.

Recently it has been concluded by the U.S. National Mastitis Council that the use of a re-milking sanitization step further decreases mastitis, and presents other benefits, such as decreasing the surface pathogen load (such as Escherichia coli and Listeria spp.) and pathogen-related toxin content of milk. Therefore, the industrial recommendation for the use of teat sanitizers presently involves both a pre- and post-milking application. The presently-recommended process of milking is therefore as follows: prior to milking, the teats of the animal to be milked are sanitized with the pre-milking sanitizer, which is then quickly wiped off with a clean towel. The animal is then milked with the automated milker. After milking, the teat is highly susceptible to infection, because the teat-end sphincter muscle (responsible for closing the teat-end) remains open for approximately 30 minutes after milking. Therefore, a post-milking sanitizer is applied and left on the skin (i.e. not rinsed off or deliberately removed) until the next milking.

While there are a number of germicides that are effective in preventing and treating mastitis, most preparations have the disadvantage of only remaining in contact with the udder for a short time due to the mobility of the preparation. Longer contact time is desirable in order to insure a higher kill rate for the harmful bacteria.

Because the teat sanitizer is left on the skin for a long period of time, the formulation must not have a tendency to irritate or damage the skin. Any toxic effects would be even more pronounced in a four-times daily milking herd, where the pre- and post-milking sanitization applications could reach up to eight times per day. Due to the difficulty in formulation of a composition which has a satisfactory antimicrobial activity but which also does not damage the skin, the majority of compositions exist in the field that are indicated for use as either a pre-milking, biocidal sanitizer, or as a post-milking biocidal sanitizer/skin conditioner. Generally, the pre-milking sanitizers contain a greater germicidal activity (usually a greater concentration of biocidal active ingredients) than post-milking sanitizers/conditioners because the pre-milking sanitizer does not remain in prolonged contact with the skin.

Active ingredients for teat sanitizer compositions include iodine; although, others have been used. Iodine is perhaps the most widely used active ingredient in such compositions, mainly due to its low cost and fairly broad antimicrobial spectrum. At concentrations allowable in milk, however, iodine has a relatively slow kill time in comparison to other popular active agents. Iodine also confers no persistence of antimicrobial activity (i.e. continued killing ability due to retention of the active ingredient in the target tissue) with continued use. Furthermore, at concentrations necessary for usefulness as a biocidal agent, iodine damages the udder skin in frequent milking situations and may not be compatible with other active antimicrobial agents used at other steps in the milking process. Even in once- to twice-daily milking situations, iodine can have a long-term negative effect on the udder skin condition, in part due to tissue denaturation, and to the formation of salts of the counter-ion with environmental anions (e.g., Cl⁻) on the skin surface after the formulation has dried.

There is therefore a long felt need for compositions for treating mastitis in an animal that has a rapid kill time, is compatible with the skin of the animal being treated and which has no long-term negative effects on the udder skin condition. The compositions and methods disclosed herein meet these and other needs.

### SUMMARY OF THE DISCLOSURE

The disclosed compositions and methods provide a method for preventing, controlling, and treating mastitis in an animal. The compositions and methods are suitable for use with any mammal, including, but not limited to, cows, goats, llamas, and the like.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### SUMMARY OF THE FIGURES

FIG. 1 is a photograph depicting cracked, dry, infected teats.

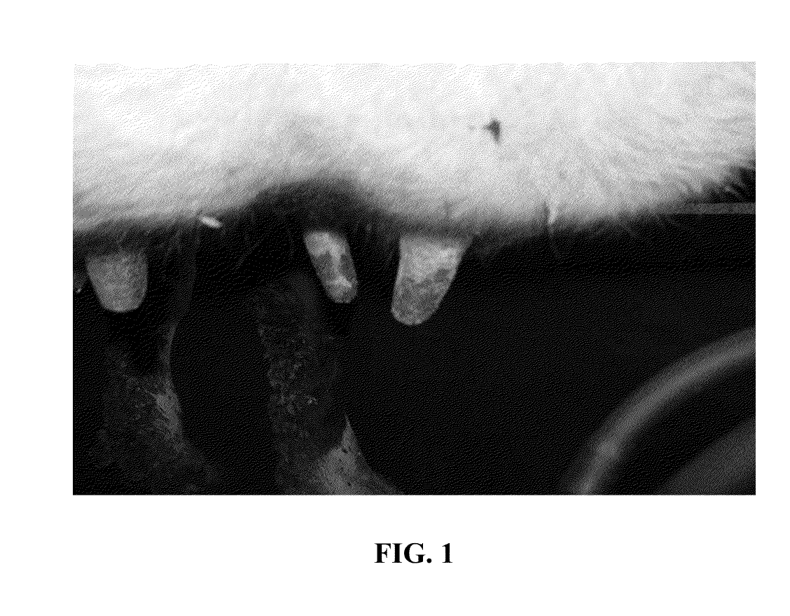

FIG. 2 is a photograph depicting soft, supple, health teats after only 2 weeks treatment with the compositions disclosed herein.

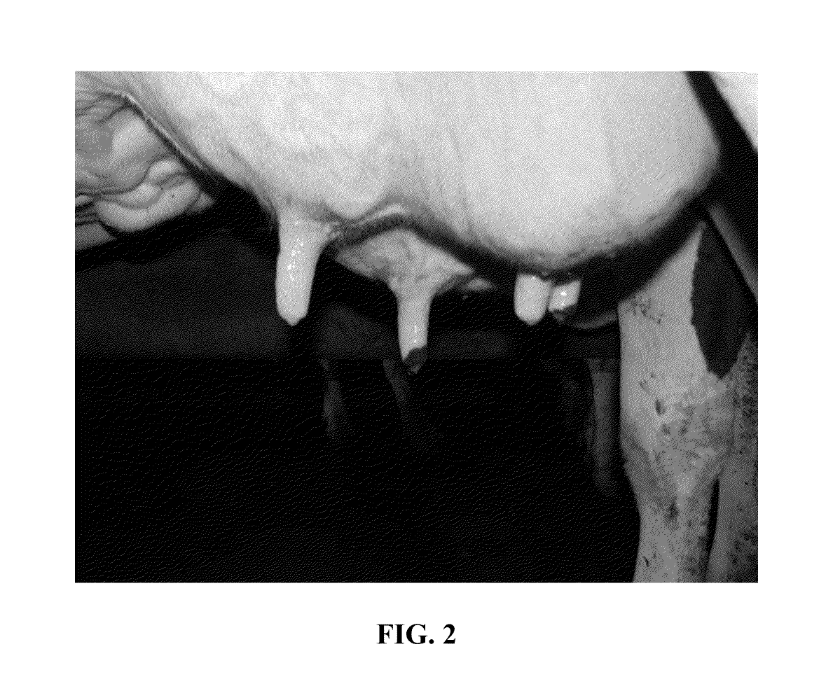

### DETAILED DISCLOSURE

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a carrier” includes mixtures of two or more such carriers, and the like.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

A weight percent of a component, unless specifically stated to the contrary, is based on the total weight of the formulation or composition in which the component is included.

By “contacting” is meant an instance of applying a composition to a mammal. Contacting can include applying a composition directly, for example, by hand, by brush, by dipping the animal, or a part or portion thereof, in a container comprising a disclosed composition, by spraying, or by applying the disclosed composition to the animal's surroundings, for example, straw, milking machine, and the like, such that the disclosed composition contacts the animal.

By “sufficient amount” and “sufficient time” means an amount and time needed to achieve the desired result or results, e.g., control and/or prevention of infection of a mammal.

“Admixture” or “blend” is generally used herein means a physical combination of two or more different components

“Controlled release” as used herein means the use of a material to regulate the release of another substance.

“Excipient” is used herein to include any other compound that may be contained in or on the microparticle that is not a therapeutically or biologically active compound. As such, an excipient should be pharmaceutically or biologically acceptable or relevant (for example, an excipient should generally be non-toxic to the subject). “Excipient” includes a single such compound and is also intended to include a plurality of excipients.

“Primary biocide” is used herein to refer to compounds that are biologically active against a primary pathogen.

“Primary pathogen” is used herein to refer to bacteria, viruses, or other biologically active microorganisms that can cause infection in a mammal.

The present disclosure addresses several unmet needs, inter alia;
1) Providing compositions effective in killing one or more pathogens, non-limiting examples of which include Streptococcus agalactiae, Staphylococcus aureous, and Mycoplasma spp.

2) Providing compositions effective in preventing the infection and/or spread of one or more pathogens from an infected animal to other animals or an apparatus that contacts an infected animal, for example, milking machines, bedding, stalls, and the like. Non-limiting examples of infection causing pathogens include: Streptococcus agalactiae, Staphylococcus aureous, and Mycoplasma spp.
3) Providing compositions effective against environmental pathogens, non-limiting examples of which include Streptococcus spp, Escherichia coli, Klebsiella species, A. pyogenes, and Pseudomonas species. In addition, the disclosed compositions are suitable for controlling yeast.

4) Providing compositions that are a replacement for iodine-based treatments.
5) Providing methods for preventing mastitis in a mammal and methods for treating mastitis in a mammal.

### Treatment Compositions

A first embodiment of the disclosed compositions for improving teat and udder hygiene in a mammal, comprises:
a) from about 0.05% to about 8% by weight of a biocidal system, comprising:
   i) at least about 75% by weight of a primary biocide; and
   ii) at least about 5% by weight of a preservative component;
b) from about 0.05% to about 0.2% by weight of a nonionic surfactant having an HLB of from about 10 to about 20;
c) from about 1% to about 3% by weight of an emollient system comprising:
   i) at least about 20% by weight of an extradermal penetrating agent; and
   ii) at least about 50% by weight of an emollient base;
d) from about 0.1% to about 1% by weight of a thickening agent; and
e) the balance an aqueous based carrier.

Another embodiment comprises:
a) from about 0.05% to about 3% by weight of a biocidal system, comprising:
   i) from about 75% to about 95% by weight of a primary biocide; and
   ii) from about 5% to about 25% by weight of a preservative component;
b) from about 0.05% to about 0.2% by weight of a nonionic surfactant having an HLB of from about 10 to about 20;
c) from about 1% to about 3% by weight of an emollient system comprising:
   i) at least about 20% by weight of an extradermal penetrating agent; and
   ii) at least about 50% by weight of an emollient base;
d) from about 0.1% to about 1% by weight of a thickening agent; and
e) the balance an aqueous based carrier.

A further embodiment comprises:
a) from about 0.05% to about 3% by weight of a biocidal system, comprising:
   i) from about 75% to about 95% by weight of cetyl pyridinium chloride; and
   ii) from about 5% to about 25% by weight of urea;
b) from about 0.05% to about 0.2% by weight of a nonionic surfactant having an HLB of from about 10 to about 20;
c) from about 1% to about 3% by weight of an emollient system comprising:
   i) at least about 20% by weight of an extradermal penetrating agent; and
   ii) at least about 50% by weight of an emollient base;
d) from about 0.1% to about 1% by weight of a thickening agent; and
e) the balance an aqueous based carrier.

A yet further embodiment comprises:
a) from about 0.05% to about 3% by weight of a biocidal system, comprising:
   i) at least about 75% by weight of a primary biocide; and
   ii) at least about 5% by weight of a preservative component;
b) from about 0.05% to about 0.2% by weight of a nonionic surfactant having an HLB of from about 10 to about 20;
c) from about 1% to about 3% by weight of an emollient system comprising:
   i) at least about 30% by weight of an extradermal penetrating agent; and
   ii) at least about 60% by weight of an emollient base;
d) from about 0.1% to about 1% by weight of a thickening agent; and
e) the balance an aqueous based carrier.

However, other non-limiting embodiments and combinations are possible as further disclosed herein.

### Biocidal System

The disclosed compositions comprise a biocidal system. The biocidal system comprises a primary biocide and a preservative. The preservative is chosen for compatibility with the primary biocide.

Suitable biocides include quaternary ammonium compounds chosen from (C₁₂-C₁₄ alkyl)(C₁-C₂ dialkyl)benzyl ammonium salts, N-(C₁₂-C₁₈ alkyl)heteroaryl ammonium salts, and N-[(C₁₂-C₁₄ alkyl)(C₁-C₂ dialkyl)]heteroarylalkylene ammonium salts. Non-limiting examples of the (C₁₂-C₁₄ alkyl)(C₁-C₂ dialkyl)benzyl ammonium salts include (C₁₂-C₁₄ alkyl)dimethyl-benzyl ammonium chloride, (C₁₂-C₁₄ alkyl)dimethylbenzyl ammonium bromide, and (C₁₂-C₁₄ alkyl)dimethylbenzyl ammonium hydrogen sulfate. Non-limiting examples of the N-(C₁₂-C₁₈ alkyl)heteroaryl ammonium salts include cetyl pyridinium chloride, cetyl pyridinium bromide, and cetyl pyridinium hydrogen sulfide. For the N-(C₁₂-C₁₈ alkyl)heteroaryl ammonium salts other anions can be used.

Further examples of quaternary ammonium compounds suitable for use as the primary biocides include cetyltrimethylammonium chloride, stearyltrimethylammonium chloride, isostearyltrimethylammonium chloride, lauryltrimethylammonium chloride, behenyltrimethyl-ammonium chloride, octadecyltrimethylammonium chloride, cocoyltriinethylammonium chloride, cetyltrimethylammonium bromide, stearyltrimethylammonium bromide, lauryl-trimethylammonium bromide, isostearyllauryldimethylammonium chloride, dicetyldimethyl-ammonium chloride, distearyldimethylammonium chloride, dicocoyldimethylammonium chloride, γ-gluconamidopropyldimethylhydroxyethylammonium chloride, di-[polyoxyethylene(2)]oleylmethylammonium chloride, dodecyldimethylethylammonium chloride, octyldihydroxyethylmethylammonium chloride, tri[polyoxyethylene(5)]-stearylammonium chloride, polyoxypropylenemethyldiethylammonium chloride, lauryl-dimethyl(ethylbenzyl)ammonium chloride, behenamidopropyl-N,N-dimethyl-N-(2,3-dihydroxypropyl)ammonium chloride, tallowdimethylammoniopropyltrimethylammonium dichloride, and benzalconium chloride.

Cetylpyridinium chloride is available from Wako Pure Chemical Industries, Ltd.

Preservatives suitable for use in the disclosed biocidal systems include urea and urea derivatives, for example, imidazolyl urea, hydantoin, dichlorodimethylhydantoin, bromochlorodimethylhydantoin, dibromodimethylhydantoin, and biuret.

Further examples of preservatives include parabens, such as the methyl and propyl parabens; urea derivatives, the cis isomer of 1-(3-chloroallyl)-3,5,7-triaza-1-azonia adamantane chloride (CFTA designation, quaternium-15); and other standard pharmaceutical grade preservatives.

The disclosed compositions comprise from about 0.05% to about 3% by weight of a biocidal system, comprising:
i) at least about 50% by weight of a primary biocide; and
ii) at least about 5% by weight of a preservative component.

One aspect of the disclosed compositions comprise:
i) from about 75% to about 95% by weight of a primary biocide; and
ii) from about 5% to about 25% by weight of a preservative component.

Another aspect of the disclosed compositions comprise:
i) from about 75% to about 90% by weight of a primary biocide; and
ii) from about 10% to about 25% by weight of a preservative component.

A yet further aspect of the disclosed compositions comprise:
i) from about 80% to about 95% by weight of a primary biocide; and
ii) from about 5% to about 20% by weight of a preservative component.

A still further aspect of the disclosed compositions comprise:
i) from about 85% to about 95% by weight of a primary biocide; and
ii) from about 5% to about 15% by weight of a preservative component

A non-limiting example of a disclosed biocidal system includes:
i) 90% by weight of cetyl pyridinium chloride; and
ii) 10% by weight of urea.

Another non-limiting example of a disclosed biocidal system includes:
i) from about 80 to about 95% by weight of cetyl pyridinium chloride; and
ii) from about 5% to about 20% by weight of urea.

A yet further non-limiting example of a disclosed biocidal system includes:
i) from about 80 to about 90% by weight of cetyl pyridinium chloride; and
ii) from about 10% to about 20% by weight of urea.

A still further non-limiting example of a disclosed biocidal system includes:
i) from about 75 to about 90% by weight of cetyl pyridinium chloride; and
ii) from about 10% to about 25% by weight of urea.

The biocidal systems disclosed herein can comprise 75%, 76%, 77%, 78%, 79%, 80%, 81%, 82%, 83%, 84%, 85%, 86%, 87%, 88%, 89%, 90%, 91%, 92%, 93%, 94%, or 95% by weight of a primary biocide.

### Surfactant

The disclosed compositions comprise from about 0.05% to about 0.2% by weight of a surfactant. In further aspect, the disclosed compositions comprise from about 0.05% to about 0.2% by weight of a non-ionic surfactant. In one embodiment the surfactant has an HLB of from about 10 to about 20. One aspect of the disclosed compositions comprises a surfactant having an HLB of from about 12 to about 18. A further aspect of the disclosed compositions comprises a surfactant having an HLB of from about 13 to about 16. Another embodiment of the disclosed compositions comprise from about 0.1% to about 0.2% by weight of a surfactant.

In one embodiment, the compositions comprise a nonionic surfactant having an

HLB of from about 10 to about 20. One aspect of the disclosed compositions comprises a nonionic surfactant having an HLB of from about 12 to about 18. A further aspect of the disclosed compositions comprises a nonionic surfactant having an HLB of from about 13 to about 16. Another embodiment of the disclosed compositions comprise from about 0.1% to about 0.2% by weight of a nonionic surfactant.

Suitable surfactants include anionic surfactants, for example, linear alkyl sulfates. Non-limiting examples of linear alkyl sulfate surfactants include C₁₀ (decyl) sulfate, C₁₂ (dodecyl) sulfate, and C₁₄ (tetradecyl) sulfate. In addition, mixtures of two or more alkyl surfactants can be used. Suitable salts of linear alkyl sulfates include ammonium, sodium, and potassium.

In addition, branched alkyl surfactants can be used in the disclosed compositions, for example, mid-chain branched alkyl sulfate surfactants as disclosed in U.S. Pat. No. 6,232,282 included herein by reference in its entirety.

Suitable nonionic surfactants for use in the disclosed compositions include polyoxyethylene C₆-C₁₂ alkylphenyl ethers, polyoxyethylene sorbitan tri(C₁₂⁻C₁₈)-alkanoates, polyoxyethylene sorbitan di(C₁₂-C₁₈)-alkanoates, polyoxyethylene sorbitan mono-, di-, and tri-(C₁₂-C₁₈)-alkanoates, and polyoxyethylene C₁₂-C₂₀ alkyl ethers. One category of suitable nonionic surfactants for use in the disclosed compositions are the polyoxyethylene C₆-C₁₂ alkylphenyl ethers having the formula:

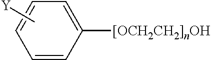

wherein Y is a C₆-C₁₂ alkyl unit and n is an index from 5 to 40. Non-limiting examples of C₆-C₁₂ alkylphenyl ethers includes polyoxyethylene(5) isooctylphenyl ethers sold under the tradenames IGEPAL™ CA-520 and IGEPAL™ CO-520, polyoxyethylene(8) isooctylphenyl ethers sold under the tradename TRITON™ X-114, polyoxyethylene(9) nonylphenyl ether sold under the tradename IGEPAL™ CO-630, polyoxyethylene(10) isooctylphenyl ether sold under the tradename TRITON™ X-100, polyoxyethylene(branched) nonylphenyl ethers sold under the tradename TRITON™ N-101, polyoxyethylene(12) nonylphenyl ether sold under the tradename IGEPAL™ CO-720, polyoxyethylene(12) isooctylphenyl ether sold under the tradename IGEPAL™ CA-720, polyoxyethylene(40) nonylphenyl ether sold under the tradename IGEPAL™ CO-890, and polyoxyethylene(40) isooctylphenyl ether sold under the tradename TRITON™ X-405.

Another category of nonionic surfactants for use in the disclosed compositions are polyoxyethylene sorbitan mono-, di-, and tri-(C₁₂-C₁₈)-alkanoates, non-limiting examples of which include polyoxyethylene(20) sorbitan trioleate sold under the tradename TWEEN™ 85, polyoxyethylene(20) sorbitan monooleate sold under the tradename TWEEN™ 80, polyoxy-ethylene(20) sorbitan monostearate sold under the tradename TWEEN™ 60, polyoxyethyl-ene(20) sorbitan monopalmitate sold under the tradename TWEEN™ 40, and polyoxyethyl-ene(20) sorbitan monolaurate sold under the tradename TWEEN™ 20.

A further category of nonionic surfactants for use in the disclosed compositions are polyoxyethylene C₉-C₂₀ alkyl ethers, non-limiting examples of which include ethoxylate alcohols having the formula:

RO(CH₂CH₂O)ₘH

wherein R is a linear or branched alkyl group having from 6 to 20 carbon atoms and m is an integer of about 2 to about 20. On example of suitable ethoxylate alcohol surfactants are the NEODOL™ ethoxylated alcohols from Shell Chemicals. Non-limiting examples of suitable ethoxylated alcohols include NEODOL™ 91-5, NEODOL™ 91-6, NEODOL™ 91-8, NEODOL™ 91-9, NEODOL™ 23-6.5, NEODOL™ 25-5, NEODOL™ 25-7, NEODOL™ 25-9, NEODOL™ 25-12, NEODOL™ 45-7, and NEODOL™ 135-7, available from BASF.

### Emollient System

The disclosed compositions comprise from about 1% to about 3% by weight of an emollient system comprising:
i) at least about 20% by weight of an extradermal penetrating agent; and
ii) at least about 50% by weight of an emollient base.

One component of the emollient system relates to extradermal penetrating agents that provide for penetration of dry or damaged teat or utter skin and functions to help carry and retain the biocidal system in contact with the affected tissue. Suitable extradermal penetrating agents include C₁-C₈ mono- or poly-hydroxy alcohols, non-limiting examples of which include benzyl alcohol, ethylene glycol, and propylene glycol. A combination of C₁-C₈ linear alcohols can also be used as the extradermal penetrating agent, however, the amount of C₁-C₈ linear alcohol is adjusted according to the type and amount of thickening agent used. This adjustment is within the scope of the artisan. One example of a suitable extradermal penetrating agent is propylene glycol.

The emollient system further comprises an emollient base. The emollient base comprises about one-half of the emollient system. Non-limiting examples of emollient bases includes C₉-C₁₄ linear or branched alkyl alcohols, C₃-C₁₄ linear or branched polyols, C₆-C₁₄ di-esters of C₆-C₁₂ diacids, hydrocarbons, natural waxes, vegetable oils, and silicones.

One embodiment of emollient bases includes polyols having the formula:

HOCH₂—[CHOH]ₓ—CH₂OH

wherein the index x is from 1 to 20.

In another iteration of polyols the index x is from 1 to 10. In a further iteration the emollient base includes polyols chosen from glycerol, (2R,3R)-butane-1,2,3,4-tetraol, (2S,3R)-butane-1,2,3,4-tetraol, (2R,3S)-butane-1,2,3,4-tetraol, (2S,35)-butane-1,2,3,4-tetraol, (2R,3R,4R)-pentane-1,2,3,4,5-pentaol, (2S,3R,4R)-pentane-1,2,3,4,5-pentaol, (2R,3S,4R)-pentane-1,2,3,4,5-pentaol, (2R,3R,4S)-pentane-1,2,3,4,5-pentaol, (2S,3S,4R)-pentane-1,2,3,4,5-pentaol, (2S,3R,4S)-pentane-1,2,3,4,5-pentaol, (2R,3S,4S)-pentane-1,2,3,4,5-pentaol, and (2S,3S,4S)-pentane-1,2,3,4,5-pentaol. In one iteration of the disclosed compositions, the emollient base is glycerol. Various polyols are also known by their common names, inter alia, erythritol and xylitol.

The emollient base can also be a combination of one or more emollient bases, for example, glycerol in combination with ethoxylated partial glyceride fatty acid esters, however, the various other emollient bases that are useful in the present composition include those compatible with the biocidal system and which promote general skin health and integrity in high frequency milking conditions. These include branched chain esters, ethoxylated partial glyceride fatty acid esters, protein derivatives, lanolin and lanolin derivatives, and fatty alcohol ethoxylates, emollient oils, fatty acids, fatty alcohols and their esters. The relative concentrations of extradermal penetrating agent and emollient base in the disclosed compositions are easily determined by those skilled in the art.

A further example of suitable emollient bases include isononyl isonanoate, dioctyl sebacate, isooctyl isooctanoate, dioctyl adipate, squalane, petrolatum, mineral oil, carnauba wax, candelilla wax, beeswax, sunflower oil, sesame oil, olive oil, cyclomethicone and dimethicone.

In a further embodiment, emollient system comprises from about 1% to about 2% by weight of an emollient system, the emollient system comprising:
i) from about 30% to about 40% by weight of an extradermal penetrating agent; and
ii) from about 60% to about 70% by weight of an emollient base.

In another embodiment, emollient system comprises from about 1% to about 2% by weight of an emollient system, the emollient system comprising:
i) from about 35% to about 45% by weight of an extradermal penetrating agent; and
ii) from about 55% to about 65% by weight of an emollient base.

In a yet further embodiment, emollient system comprises from about 1% to about 2% by weight of an emollient system, the emollient system comprising:
i) from about 25% to about 40% by weight of an extradermal penetrating agent; and
ii) from about 60% to about 75% by weight of an emollient base.

In a still further embodiment, emollient system comprises from about 1% to about 2% by weight of an emollient system, the emollient system comprising:
i) from about 30% to about 40% by weight of an extradermal penetrating agent; and
ii) from about 60% to about 70% by weight of an emollient base.

The compositions disclosed herein can comprise 1%, 2%, or 3% or an emollient system, or any fractional part thereof, for example 1.1%, 1.2%, 1.3%, 1.4%, 1.5%, 1.6%, 1.7%, 1.8%, and 1.9%.

### Thickening Agent

The disclosed compositions further comprise from about 0.1% to about 1% by weight of a thickening agent. Suitable thickening agents include hydroxymethyl cellulose, hydroxyethyl cellulose, methylcellulose, hydroxypropyl cellulose, methyl cellulose, carboxy methylcellulose, emulsifying waxes, alkyl triammonium methosulfate, and ceteraryl octanoate. Although the disclosed compositions are aqueous based, certain ingredients may require the presence of a more lipophilic solvent for proper stabilization. Preferred additional solvents are polyhydric alcohol solvents, or “polyol” solvents, such as the polyalkylene glycols having alkylene moieties containing about 2-3 carbon atoms, preferably the polyethylene glycols. Molecular weight ranges of from about 200-4000 are preferred for the polyalkylene glycols (e.g., propylene glycol).

One embodiment of the disclosed compositions, utilizes hydroxyethyl cellulose in amounts of 0.5%, 0.6%, 0.7%, 0.8%, 0.9%, and 1% by weight of the composition adjusted for the emollient system and for the final method of applying the composition to the animal in need of treatment.

In a further embodiment, the thickener can be hydroxymethyl cellulose in amounts of 0.5%, 0.6%, 0.7%, 0.8%, 0.9%, and 1% by weight of the composition adjusted for the emollient system and for the final method of applying the composition to the animal in need of treatment.

In a further embodiment, the thickener can be hydroxyethyl cellulose in amounts of 0.5%, 0.6%, 0.7%, 0.8%, 0.9%, and 1% by weight of the composition adjusted for the emollient system and for the final method of applying the composition to the animal in need of treatment.

In yet a further embodiment, the thickener can be hydroxypropyl cellulose in amounts of 0.5%, 0.6%, 0.7%, 0.8%, 0.9%, and 1% by weight of the composition adjusted for the emollient system and for the final method of applying the composition to the animal in need of treatment.

Another aspect of the thickening agents relates to agents that are natural gums. Non-limiting examples of natural gums include Guar gum, Xanthan gum, Locust Bean Gum, Gum Arabic, and Carrageenan.

One embodiment of the disclosed compositions, utilizes Guar gum in amounts of 0.5%, 0.6%, 0.7%, 0.8%, 0.9%, and 1% by weight of the composition adjusted for the emollient system and for the final method of applying the composition to the animal in need of treatment.

In a further embodiment, the thickener can be Xanthan gum in amounts of 0.5%, 0.6%, 0.7%, 0.8%, 0.9%, and 1% by weight of the composition adjusted for the emollient system and for the final method of applying the composition to the animal in need of treatment.

In a further embodiment, the thickener can be Locust Bean Gum in amounts of 0.5%, 0.6%, 0.7%, 0.8%, 0.9%, and 1% by weight of the composition adjusted for the emollient system and for the final method of applying the composition to the animal in need of treatment.

In yet a further embodiment, the thickener can be Gum Arabic in amounts of 0.5%, 0.6%, 0.7%, 0.8%, 0.9%, and 1% by weight of the composition adjusted for the emollient system and for the final method of applying the composition to the animal in need of treatment.

In yet a further embodiment, the thickener can be Carrageenan in amounts of 0.5%, 0.6%, 0.7%, 0.8%, 0.9%, and 1% by weight of the composition adjusted for the emollient system and for the final method of applying the composition to the animal in need of treatment.

### Carriers

The balance of the disclosed compositions comprises a carrier. The carrier can be any suitable material that can dissolve the active ingredients and co-ingredients and deliver the biocidal system to the infected areas of the animal being treated. Water is a convenient carrier for liquid embodiments of the disclosed composition. However, alcohols can be used to assist in the dissolving of the ingredients prior to dilution with water. Embodiments of the disclosed compositions include gels, salves, and creams, especially for treating cases wherein the infection may be chronic and the animal must be isolated from the rest of the animals and given more intense treatment.

### Adjunct Ingredients

The disclosed compositions can further comprise one or more dyes at levels of from about 0.001% to 0.5%. Non-limiting examples of suitable dyes are Alizarine Light Blue B (C.I. 63010), Carta Blue VP (C.I. 24401), Acid Green 2G (C.I. 42085), Astrogen Green D (C.I. 42040), Supranol Cyanine 7B (C.I. 42675, Maxilon Blue 3RL (C.I. Basic Blue 80), Drimarine Blue Z-RL (C.I. Reactive Blue 18), Alizarine Light Blue H-RL (C.I. Acid Blue 182), FD&C Blue No. 1 and FD&C Green No. 3. (See U.S. Pat. No. 4,248,827 and U.S. Pat. No. 4,200,606, both incorporated herein by reference.) C.I. refers to Color Index.

Another adjunct ingredient suitable for use in the compositions disclosed herein includes fragrances, for example, fragrances as disclosed in U.S. Pat. No. 6,013,618 included herein by reference in its entirety.

### Formulations

The following are non-limiting examples of the disclosed compositions. The following formulations can serve either as a spray-on, wipe-on, or as a dip, however, the formulator can choose any convenient method of applying the following formulations.

TABLE I

| Ingredients | 1 | 2 | 3 | 4 | 5 |
|---|---|---|---|---|---|
| cetyl pyridinium chloride | 0.135 | 0.130 | 0.125 | 0.12 | 0.115 |
| urea | 0.015 | 0.02 | 0.025 | 0.03 | 0.035 |
| TRITON X-100 | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| hydroxyethylcellulose | 0.5 | 0.5 | 0.5 | 0.5 | 0.5 |
| carrier | Balance | Balance | Balance | Balance | Balance |

TABLE II

| Ingredients | 6 | 7 | 8 | 9 | 10 |
|---|---|---|---|---|---|
| cetyl pyridinium chloride | 0.125 | 0.135 | 0.2 | 0.2 | 0.2 |
| urea | — | — | 0.04 | 0.03 | 0.02 |
| imidazolyl urea | 0.02 | 0.015 | — | — | — |
| TRITON X-100 | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| hydroxyethylcellulose | 0.5 | 0.5 | 0.5 | 0.5 | 0.5 |
| carrier | Balance | Balance | Balance | Balance | Balance |

TABLE III

| Ingredients | 11 | 12 | 13 | 14 | 15 |
|---|---|---|---|---|---|
| Cetyl pyridinium | 0.135 | 0.130 | 0.125 | 0.12 | 0.115 |
| chloride | | | | | |
| urea | 0.015 | 0.02 | 0.025 | 0.03 | 0.035 |
| TRITON X-100 | 0.2 | 0.2 | 0.2 | 0.2 | 0.2 |
| propylene glycol | 0.5 | 0.75 | 1.0 | 0.5 | 0.75 |
| glycerol | 2.0 | 2.0 | 2.0 | 2.0 | 2.0 |
| hydroxyethylcellulose | 0.5 | 0.5 | 0.5 | 0.5 | 0.5 |
| carrier | Balance | Balance | Balance | Balance | Balance |

TABLE IV

| Ingredients | 16 | 17 | 18 | 19 | 20 |
|---|---|---|---|---|---|
| cetyl pyridinium chloride | 0.135 | 0.130 | 0.125 | 0.12 | 0.115 |
| urea | 0.015 | 0.02 | 0.025 | 0.03 | 0.035 |
| TRITON X-100 | 0.2 | 0.2 | 0.2 | 0.2 | 0.2 |
| propylene glycol | 0.25 | 0.15 | 0.5 | 1.5 | 1.0 |
| glycerol | 0.95 | 1.25 | 1.0 | 1.5 | 1.5 |
| hydroxyethylcellulose | 0.5 | 0.5 | 0.5 | 0.5 | 0.5 |
| carrier | Balance | balance | balance | balance | balance |

TABLE V

| Ingredients | 21 | 22 | 23 | 24 | 25 |
|---|---|---|---|---|---|
| cetyl pyridinium chloride | 0.135 | 0.130 | 0.125 | 0.12 | 0.115 |
| urea | 0.015 | 0.02 | 0.025 | 0.03 | 0.035 |
| TRITON X-100 | 0.2 | 0.2 | 0.2 | 0.2 | 0.2 |
| propylene glycol | 0.5 | 0.75 | 1.0 | 0.5 | 0.75 |
| glycerol | 2.0 | 2.0 | 2.0 | 2.0 | 2.0 |
| hydroxyethylcellulose | 0.1 | 0.25 | 0.4 | 0.75 | 1.0 |
| carrier | Balance | Balance | Balance | Balance | Balance |

TABLE VI

| Ingredients | 26 | 27 | 28 | 29 | 30 |
|---|---|---|---|---|---|
| (C₁₂-C₁₄ alkyl)- | 0.135 | 0.130 | 0.125 | 0.12 | 0.115 |
| dimethylbenzyl | | | | | |
| ammonium chloride | | | | | |
| urea | 0.015 | 0.02 | 0.025 | 0.03 | 0.035 |
| TRITON X-100 | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| hydroxyethylcellulose | 0.5 | 0.5 | 0.5 | 0.5 | 0.5 |
| carrier | Balance | Balance | Balance | Balance | Balance |

TABLE VII

| Ingredients | 31 | 32 | 33 | 34 | 35 |
|---|---|---|---|---|---|
| (C₁₂-C₁₄ alkyl)- | 0.125 | 0.135 | 0.2 | 0.2 | 0.2 |
| dimethylbenzyl | | | | | |
| ammonium chloride | | | | | |
| urea | — | — | 0.04 | 0.03 | 0.02 |
| imidazolyl urea | 0.02 | 0.015 | — | — | — |
| TRITON X-100 | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| hydroxyethylcellulose | 0.5 | 0.5 | 0.5 | 0.5 | 0.5 |
| carrier | Balance | Balance | Balance | Balance | Balance |

TABLE VIII

| Ingredients | 36 | 37 | 38 | 39 | 40 |
|---|---|---|---|---|---|
| cetyl pyridinium chloride | 0.135 | 0.130 | 0.125 | 0.12 | 0.115 |
| urea | 0.015 | 0.02 | 0.025 | 0.03 | 0.035 |
| TRITON N-101 | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| hydroxyethylcellulose | 0.5 | 0.5 | 0.5 | 0.5 | 0.5 |
| carrier | Balance | Balance | Balance | Balance | Balance |

TABLE IX

| Ingredients | 41 | 42 | 43 | 44 | 45 |
|---|---|---|---|---|---|
| cetyl pyridinium chloride | 0.125 | 0.135 | 0.2 | 0.2 | 0.2 |
| urea | — | — | 0.04 | 0.03 | 0.02 |
| imidazolyl urea | 0.02 | 0.015 | — | — | — |
| TRITON N-101 | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| hydroxyethylcellulose | 0.5 | 0.5 | 0.5 | 0.5 | 0.5 |
| carrier | Balance | Balance | Balance | Balance | Balance |

TABLE X

| Ingredients | 46 | 47 | 48 | 49 | 50 |
|---|---|---|---|---|---|
| cetyl pyridinium chloride | 0.135 | 0.130 | 0.125 | 0.12 | 0.115 |
| Urea | 0.015 | 0.02 | 0.025 | 0.03 | 0.035 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| guar gum | 0.5 | 0.5 | 0.5 | 0.5 | 0.5 |
| carrier | Balance | Balance | Balance | Balance | Balance |

TABLE XI

| Ingredients | 51 | 52 | 53 | 54 | 55 |
|---|---|---|---|---|---|
| cetyl pyridinium chloride | 0.135 | 0.130 | 0.125 | 0.12 | 0.115 |
| urea | 0.015 | 0.02 | 0.025 | 0.03 | 0.035 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| guar gum | 0.2 | 0.2 | 0.2 | 0.2 | 0.2 |
| carrier | Balance | Balance | Balance | Balance | Balance |

TABLE XII

| Ingredients | 56 | 57 | 58 | 59 | 60 |
|---|---|---|---|---|---|
| cetyl pyridinium chloride | 0.135 | 0.130 | 0.125 | 0.12 | 0.115 |
| urea | 0.015 | 0.02 | 0.025 | 0.03 | 0.035 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| xanthan gum | 0.5 | 0.5 | 0.5 | 0.5 | 0.5 |
| carrier | Balance | Balance | Balance | Balance | Balance |

TABLE XIII

| Ingredients | 61 | 62 | 63 | 64 | 65 |
|---|---|---|---|---|---|
| cetyl pyridinium chloride | 0.135 | 0.130 | 0.125 | 0.12 | 0.115 |
| urea | 0.015 | 0.02 | 0.025 | 0.03 | 0.035 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| xanthan gum | 0.2 | 0.2 | 0.2 | 0.2 | 0.2 |
| carrier | Balance | Balance | Balance | Balance | Balance |

TABLE XIV

| Ingredients | 66 | 67 | 68 | 69 | 70 |
|---|---|---|---|---|---|
| cetyl pyridinium chloride | 0.135 | 0.130 | 0.125 | 0.12 | 0.115 |
| urea | 0.015 | 0.02 | 0.025 | 0.03 | 0.035 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| locust bean gum | 0.5 | 0.5 | 0.5 | 0.5 | 0.5 |
| carrier | Balance | Balance | Balance | Balance | Balance |

TABLE XV

| Ingredients | 71 | 72 | 73 | 74 | 75 |
|---|---|---|---|---|---|
| cetyl pyridinium chloride | 0.135 | 0.130 | 0.125 | 0.12 | 0.115 |
| urea | 0.015 | 0.02 | 0.025 | 0.03 | 0.035 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| locust bean gum | 0.2 | 0.2 | 0.2 | 0.2 | 0.2 |
| carrier | Balance | Balance | Balance | Balance | Balance |

TABLE XVI

| Ingredients | 76 | 77 | 78 | 79 | 80 |
|---|---|---|---|---|---|
| cetyl pyridinium chloride | 0.135 | 0.130 | 0.125 | 0.12 | 0.115 |
| urea | 0.015 | 0.02 | 0.025 | 0.03 | 0.035 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| karaya gum | 0.5 | 0.5 | 0.5 | 0.5 | 0.5 |
| carrier | Balance | Balance | Balance | Balance | Balance |

TABLE XVII

| Ingredients | 81 | 82 | 83 | 84 | 85 |
|---|---|---|---|---|---|
| cetyl pyridinium chloride | 0.135 | 0.130 | 0.125 | 0.12 | 0.115 |
| urea | 0.015 | 0.02 | 0.025 | 0.03 | 0.035 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| uaraya gum | 0.2 | 0.2 | 0.2 | 0.2 | 0.2 |
| carrier | Balance | Balance | Balance | Balance | Balance |

TABLE XVIII

| Ingredients | 86 | 87 | 88 | 89 | 90 |
|---|---|---|---|---|---|
| cetyl pyridinium chloride | 0.125 | 0.135 | 0.2 | 0.2 | 0.2 |
| urea | — | — | 0.04 | 0.03 | 0.02 |
| imidazolyl urea | 0.02 | 0.015 | — | — | — |
| ammonium C₁₂ sulfate | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| gum Arabic | 0.5 | 0.5 | 0.5 | 0.5 | 0.5 |
| carrier | Balance | Balance | Balance | Balance | Balance |

TABLE XIX

| Ingredients | 91 | 92 | 93 | 94 | 95 |
|---|---|---|---|---|---|
| cetyl pyridinium chloride | 0.135 | 0.130 | 0.125 | 0.12 | 0.115 |
| urea | 0.015 | 0.02 | 0.025 | 0.03 | 0.035 |
| ammonium C₁₂ sulfate | 0.2 | 0.2 | 0.2 | 0.2 | 0.2 |
| propylene glycol | 0.5 | 0.75 | 1.0 | 0.5 | 0.75 |
| glycerol | 2.0 | 2.0 | 2.0 | 2.0 | 2.0 |
| gum Arabic | 0.2 | 02 | 0.2 | 0.2 | 0.2 |
| carrier | Balance | Balance | Balance | Balance | Balance |

TABLE XX

| Ingredients | 96 | 97 | 98 | 99 | 100 |
|---|---|---|---|---|---|
| cetyl pyridinium chloride | 0.135 | 0.130 | 0.125 | 0.12 | 0.115 |
| urea | 0.015 | 0.02 | 0.025 | 0.03 | 0.035 |
| ammonium C₁₂ sulfate | 0.2 | 0.2 | 0.2 | 0.2 | 0.2 |
| propylene glycol | 0.25 | 0.15 | 0.5 | 1.5 | 1.0 |
| glycerol | 0.95 | 1.25 | 1.0 | 1.5 | 1.5 |
| carageenan | 0.5 | 0.5 | 0.5 | 0.5 | 0.5 |
| carrier | Balance | Balance | Balance | Balance | Balance |

TABLE XXI

| Ingredients | 101 | 102 | 103 | 104 | 105 |
|---|---|---|---|---|---|
| cetyl pyridinium chloride | 2.85 | 2.8 | 2.75 | 2.7 | 2.65 |
| urea | 0.15 | 0.2 | 0.25 | 0.3 | 0.35 |
| TRITON X-100 | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| hydroxyethylcellulose | 0.5 | 0.5 | 0.5 | 0.5 | 0.5 |
| carrier | Balance | Balance | Balance | Balance | Balance |

TABLE XXII

| Ingredients | 106 | 107 | 108 | 109 | 110 |
|---|---|---|---|---|---|
| cetyl pyridinium chloride | 2.6 | 2.55 | 2.6 | 2.55 | 2.5 |
| urea | — | — | 0.4 | 0.45 | 0.5 |
| imidazolyl urea | 0.3 | 0.45 | — | — | — |
| TRITON X-100 | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| hydroxyethylcellulose | 0.5 | 0.5 | 0.5 | 0.5 | 0.5 |
| carrier | Balance | Balance | Balance | Balance | Balance |

TABLE XXIII

| Ingredients | 111 | 112 | 113 | 114 | 115 |
|---|---|---|---|---|---|
| cetyl pyridinium chloride | 2.0 | 2.1 | 2.2 | 2.3 | 2.4 |
| urea | 1.0 | 0.9 | 0.8 | 0.7 | 0.6 |
| TRITON X-100 | 0.2 | 0.2 | 0.2 | 0.2 | 0.2 |
| propylene glycol | 0.5 | 0.75 | 1.0 | 0.5 | 0.75 |
| glycerol | 2.0 | 2.0 | 2.0 | 2.0 | 2.0 |
| hydroxyethylcellulose | 0.5 | 0.5 | 0.5 | 0.5 | 0.5 |
| carrier | Balance | Balance | Balance | Balance | Balance |

TABLE XXIV

| Ingredients | 116 | 117 | 118 | 119 | 120 |
|---|---|---|---|---|---|
| cetyl pyridinium chloride | 0.75 | 0.8 | 0.9 | 0.95 | 1.0 |
| urea | 0.015 | 0.02 | 0.025 | 0.03 | 0.035 |
| TRITON X-100 | 0.2 | 0.2 | 0.2 | 0.2 | 0.2 |
| propylene glycol | 0.25 | 0.15 | 0.5 | 1.5 | 1.0 |
| glycerol | 0.95 | 1.25 | 1.0 | 1.5 | 1.5 |
| hydroxyethylcellulose | 0.5 | 0.5 | 0.5 | 0.5 | 0.5 |
| carrier | Balance | balance | balance | balance | balance |

TABLE XXV

| Ingredients | 121 | 122 | 123 | 124 | 125 |
|---|---|---|---|---|---|
| cetyl pyridinium chloride | 1.35 | 1.30 | 1.25 | 1.2 | 1.15 |
| urea | 0.15 | 0.2 | 0.25 | 0.3 | 0.35 |
| TRITON X-100 | 0.2 | 0.2 | 0.2 | 0.2 | 0.2 |
| propylene glycol | 0.5 | 0.75 | 1.0 | 0.5 | 0.75 |
| glycerol | 2.0 | 2.0 | 2.0 | 2.0 | 2.0 |
| hydroxyethylcellulose | 0.1 | 0.25 | 0.4 | 0.75 | 1.0 |
| carrier | Balance | Balance | Balance | Balance | Balance |

TABLE XXVI

| Ingredients | 126 | 127 | 128 | 129 | 130 |
|---|---|---|---|---|---|
| (C₁₂-C₁₄ alkyl)- | 1.35 | 1.30 | 1.25 | 1.2 | 1.15 |
| dimethylbenzyl | | | | | |
| ammonium chloride | | | | | |
| urea | 0.15 | 0.2 | 0.25 | 0.3 | 0.35 |
| TRITON X-100 | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| hydroxyethylcellulose | 0.5 | 0.5 | 0.5 | 0.5 | 0.5 |
| carrier | Balance | Balance | Balance | Balance | Balance |

TABLE XXVII

| Ingredients | 131 | 132 | 133 | 134 | 135 |
|---|---|---|---|---|---|
| (C₁₂-C₁₄ alkyl)- | 1.25 | 1.35 | 2.0 | 2.0 | 2.0 |
| dimethylbenzyl | | | | | |
| ammonium chloride | | | | | |
| urea | — | — | 0.4 | 0.3 | 0.2 |
| imidazolyl urea | 0.2 | 0.15 | — | — | — |
| TRITON X-100 | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| hydroxyethylcellulose | 0.5 | 0.5 | 0.5 | 0.5 | 0.5 |
| carrier | Balance | Balance | Balance | Balance | Balance |

TABLE XXVIII

| Ingredients | 136 | 137 | 138 | 139 | 140 |
|---|---|---|---|---|---|
| cetyl pyridinium chloride | 1.35 | 1.30 | 1.25 | 1.2 | 1.15 |
| urea | 0.15 | 0.2 | 0.25 | 0.3 | 0.35 |
| TRITON N-101 | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| hydroxyethylcellulose | 0.5 | 0.5 | 0.5 | 0.5 | 0.5 |
| carrier | Balance | Balance | Balance | Balance | Balance |

TABLE XXIX

| Ingredients | 141 | 142 | 143 | 144 | 145 |
|---|---|---|---|---|---|
| cetyl pyridinium chloride | 1.25 | 1.35 | 2.0 | 2.0 | 2.0 |
| urea | — | — | 0.4 | 0.3 | 0.2 |
| imidazolyl urea | 0.02 | 0.015 | — | — | — |
| TRITON N-101 | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| hydroxyethylcellulose | 0.5 | 0.5 | 0.5 | 0.5 | 0.5 |
| carrier | Balance | Balance | Balance | Balance | Balance |

TABLE XXX

| Ingredients | 146 | 147 | 148 | 149 | 150 |
|---|---|---|---|---|---|
| cetyl pyridinium chloride | 1.35 | 1.30 | 1.25 | 1.2 | 1.15 |
| Urea | 0.15 | 0.2 | 0.25 | 0.3 | 0.35 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| guar gum | 0.5 | 0.5 | 0.5 | 0.5 | 0.5 |
| carrier | Balance | Balance | Balance | Balance | Balance |

TABLE XXXI

| Ingredients | 151 | 152 | 153 | 154 | 155 |
|---|---|---|---|---|---|
| cetyl pyridinium chloride | 1.35 | 1.30 | 1.25 | 1.2 | 1.15 |
| urea | 0.15 | 0.2 | 0.25 | 0.3 | 0.35 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| guar gum | 0.2 | 0.2 | 0.2 | 0.2 | 0.2 |
| carrier | Balance | Balance | Balance | Balance | Balance |

TABLE XXXII

| Ingredients | 156 | 157 | 158 | 159 | 160 |
|---|---|---|---|---|---|
| cetyl pyridinium chloride | 2.35 | 2.30 | 2.25 | 2.2 | 2.15 |
| urea | 0.15 | 0.2 | 0.25 | 0.3 | 0.35 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| xanthan gum | 0.5 | 0.5 | 0.5 | 0.5 | 0.5 |
| carrier | Balance | Balance | Balance | Balance | Balance |

TABLE XXXIII

| Ingredients | 161 | 162 | 163 | 164 | 165 |
|---|---|---|---|---|---|
| cetyl pyridinium chloride | 2.35 | 2.30 | 2.25 | 2.2 | 2.15 |
| urea | 0.15 | 0.2 | 0.25 | 0.3 | 0.35 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| xanthan gum | 0.2 | 0.2 | 0.2 | 0.2 | 0.2 |
| carrier | Balance | Balance | Balance | Balance | Balance |

TABLE XXXIV

| Ingredients | 166 | 167 | 168 | 169 | 170 |
|---|---|---|---|---|---|
| cetyl pyridinium chloride | 1.35 | 1.30 | 1.25 | 1.2 | 1.15 |
| urea | 0.15 | 0.2 | 0.25 | 0.3 | 0.35 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| locust bean gum | 0.5 | 0.5 | 0.5 | 0.5 | 0.5 |
| carrier | Balance | Balance | Balance | Balance | Balance |

TABLE XXXV

| Ingredients | 171 | 172 | 173 | 174 | 175 |
|---|---|---|---|---|---|
| cetyl pyridinium chloride | 1.35 | 1.30 | 1.25 | 1.2 | 1.15 |
| urea | 0.15 | 0.2 | 0.25 | 0.3 | 0.35 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| locust bean gum | 0.2 | 0.2 | 0.2 | 0.2 | 0.2 |
| carrier | Balance | Balance | Balance | Balance | Balance |

TABLE XXXVI

| Ingredients | 176 | 177 | 178 | 179 | 180 |
|---|---|---|---|---|---|
| cetyl pyridinium chloride | 1.35 | 1.30 | 1.25 | 1.2 | 1.15 |
| urea | 0.15 | 0.2 | 0.25 | 0.3 | 0.35 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| karaya gum | 0.5 | 0.5 | 0.5 | 0.5 | 0.5 |
| carrier | Balance | Balance | Balance | Balance | Balance |

TABLE XXXVII

| Ingredients | 181 | 182 | 183 | 184 | 185 |
|---|---|---|---|---|---|
| cetyl pyridinium chloride | 1.35 | 1.30 | 1.25 | 1.2 | 1.15 |
| urea | 0.15 | 0.2 | 0.25 | 0.3 | 0.35 |
| ammonium C₁₂ sulfate | 0.5 | 0.5 | 0.5 | 0.5 | 0.5 |
| propylene glycol | 1.0 | 1.0 | 1.0 | 1.0 | 1.0 |
| glycerol | 2.0 | 2.0 | 2.0 | 2.0 | 2.0 |
| uaraya gum | 0.2 | 0.2 | 0.2 | 0.2 | 0.2 |
| carrier | Balance | Balance | Balance | Balance | Balance |

TABLE XXXVIII

| Ingredients | 186 | 187 | 188 | 189 | 190 |
|---|---|---|---|---|---|
| cetyl pyridinium chloride | 2.25 | 2.35 | 2.4 | 2.4 | 2.4 |
| urea | — | — | 0.4 | 0.3 | 0.2 |
| imidazolyl urea | 0.2 | 0.15 | — | — | — |
| ammonium C₁₂ sulfate | 0.2 | 0.2 | 0.2 | 0.2 | 0.2 |
| propylene glycol | 1.0 | 1.0 | 1.0 | 1.0 | 1.0 |
| glycerol | 2.0 | 2.0 | 2.0 | 2.0 | 2.0 |
| gum Arabic | 0.5 | 0.5 | 0.5 | 0.5 | 0.5 |
| carrier | Balance | Balance | Balance | Balance | Balance |

TABLE XXXIX

| Ingredients | 191 | 192 | 193 | 194 | 195 |
|---|---|---|---|---|---|
| cetyl pyridinium chloride | 1.35 | 1.30 | 1.25 | 1.2 | 1.15 |
| urea | 0.15 | 0.2 | 0.25 | 0.3 | 0.35 |
| ammonium C₁₂ sulfate | 0.2 | 0.2 | 0.2 | 0.2 | 0.2 |
| propylene glycol | 0.5 | 0.75 | 1.0 | 0.5 | 0.75 |
| glycerol | 2.0 | 2.0 | 2.0 | 2.0 | 2.0 |
| gum Arabic | 0.2 | 02 | 0.2 | 0.2 | 0.2 |
| carrier | Balance | Balance | Balance | Balance | Balance |

TABLE XL

| Ingredients | 196 | 197 | 198 | 199 | 200 |
|---|---|---|---|---|---|
| cetyl pyridinium chloride | 1.35 | 1.30 | 1.25 | 1.2 | 1.15 |
| urea | 0.15 | 0.2 | 0.25 | 0.3 | 0.35 |
| ammonium C₁₂ sulfate | 0.2 | 0.2 | 0.2 | 0.2 | 0.2 |
| propylene glycol | 0.25 | 0.15 | 0.5 | 1.5 | 1.0 |
| glycerol | 0.95 | 1.25 | 1.0 | 1.5 | 1.5 |
| carageenan | 0.5 | 0.5 | 0.5 | 0.5 | 0.5 |
| carrier | Balance | Balance | Balance | Balance | Balance |

### Methods of Use

The disclosed compositions can be used for various applications with the application route and dosage regimen are dictated by the frequency of milking and/or the skin condition of the animal. As an example of possible applications of the invention, the compositions can be used in mammals as a pre- and post-milking application to decrease the potential for mastitis, and/or subcutaneous dermatological pathologies stemming from microbial infections. An example of this includes administering the compositions to mammalian skin, specifically the udder and teats of milking animals. The composition can be applied as a cleanser, scrub (cleanser with abrasive properties), lotion, or gel. The compositions can also be used in a therapeutic manner. For example, the compositions can be used both as a cleanser or a scrub composition to help heal udder and teat skin which has been damaged by frequent milking. Additional applications for the sanitizer include vaginal cleansers, calving sanitizers, burn disinfectants, wound healing aids, and perianal and colostomy wipe applications. For wipes, the formulation of the present invention may be applied to paper or cloth towelettes.

Disclosed herein are methods for treating mastitis and/or improving teat and udder hygiene in an animal comprising, contacting an animal with an effective amount of a disclosed composition. In one aspect, the method comprises contacting one or more teats of an infected animal with a composition comprising:
a) from about 0.05% to about 8% by weight of a biocidal system, comprising:
   i) at least about 50% by weight of a primary biocide; and
   ii) at least about 5% by weight of a preservative component;
b) from about 0.05% to about 0.2% by weight of a surfactant;
c) from about 1% to about 3% by weight of an emollient system comprising:
   i) at least about 20% by weight of an extradermal penetrating agent; and
   ii) at least about 50% by weight of an emollient base;
d) from about 0.1% to about 1% by weight of a thickening agent; and
e) the balance an aqueous based carrier.

In another aspect, the method comprises contacting one or more teats of an infected animal with a composition comprising:
a) from about 0.05% to about 3% by weight of a biocidal system, comprising:
   i) at least about 75% by weight of a primary biocide; and
   ii) at least about 5% by weight of a preservative component;
b) from about 0.05% to about 0.2% by weight of a surfactant having an HLB of from about 10 to about 20;
c) from about 1% to about 3% by weight of an emollient system comprising:
   i) at least about 20% by weight of an extradermal penetrating agent; and
   ii) at least about 50% by weight of an emollient base;
d) from about 0.1% to about 1% by weight of a thickening agent; and
e) the balance an aqueous based carrier.

In a further aspect, the method comprises contacting one or more teats of an infected animal with a composition comprising:
a) about 3% by weight of a biocidal system, comprising:
   i) 90% by weight of cetyl pyridinium chloride; and
   ii) 10% by weight of urea;
b) about 0.2% by weight of polyoxyethylene(10) isooctylphenyl ether;
c) about 3% by weight of an emollient system comprising:
   i) about 33.3% by weight of propylene glycol; and
   ii) about 66.7% by weight of glycerol;
d) about 0.5% by weight of hydroxyethylcellulose; and
e) the balance water.

In a still further aspect, the method comprises contacting one or more teats of an infected animal with a composition comprising:
a) from about 0.05% to about 3% by weight of a biocidal system, comprising:
   i) at least about 75% by weight of a primary biocide; and
   ii) at least about 5% by weight of a preservative component;
b) from about 0.05% to about 0.2% by weight of a nonionic surfactant having an HLB of from about 10 to about 20;
c) from about 1% to about 3% by weight of an emollient system comprising:
   i) at least about 20% by weight of an extradermal penetrating agent; and
   ii) at least about 50% by weight of an emollient base;
d) from about 0.1% to about 1% by weight of a thickening agent; and
e) the balance an aqueous based carrier.

In another yet aspect, the method comprises contacting one or more teats of an infected animal with a composition comprising:
a) from about 0.05% to about 3% by weight of a biocidal system, comprising:
   i) at least about 50% by weight of a primary biocide; and
   ii) at least about 10% by weight of a preservative component;
b) from about 0.05% to about 0.2% by weight of a linear alkyl sulfate;
c) from about 1% to about 3% by weight of an emollient system comprising:
   i) at least about 20% by weight of an extradermal penetrating agent; and
   ii) at least about 50% by weight of an emollient base;
d) from about 0.1% to about 1% by weight of a thickening agent chosen from Guar gum, Xanthan gum, Locust Bean Gum, Gum Arabic, and Carrageenan; and
e) the balance an aqueous based carrier.

In a yet still further aspect, the method comprises contacting one or more teats of an infected animal with a composition comprising:
a) from about 1% to about 2% by weight of a biocidal system, comprising:
   i) 90% by weight of cetyl pyridinium chloride; and
   ii) 10% by weight of urea;
b) about 0.15% by weight of a linear alkyl sulfate;
c) about 3% by weight of an emollient system comprising:
   i) about 33.3% by weight of propylene glycol; and
   ii) about 66.7% by weight of glycerol;
d) about 0.5% by weight of a thickening agent chosen from Guar gum, Xanthan gum, Locust Bean Gum, Gum Arabic, and Carrageenan; and
e) the balance water.

In a yet further aspect, the method comprises contacting one or more teats of an infected animal with a composition comprising:
a) from about 1% to about 2% by weight of a biocidal system, comprising:
   i) 90% by weight of cetyl pyridinium chloride; and
   ii) 10% by weight of urea;
b) about 0.15% by weight of a linear alkyl sulfate;
c) about 3% by weight of an emollient system comprising:
   i) about 33.3% by weight of propylene glycol; and
   ii) about 66.7% by weight of glycerol;
d) about 0.2% by weight of a thickening agent chosen from Guar gum, Xanthan gum, Locust Bean Gum, Gum Arabic, and Carrageenan; and
e) the balance water.

Disclosed herein is the use of a composition comprising:
a) from about 0.05% to about 3% by weight of a biocidal system, comprising:
   i) at least about 50% by weight of a primary biocide; and
   ii) at least about 5% by weight of a preservative component;
b) from about 0.05% to about 0.2% by weight of a surfactant;
c) from about 1% to about 3% by weight of an emollient system comprising:
   i) at least about 20% by weight of an extradermal penetrating agent; and
   ii) at least about 50% by weight of an emollient base;
d) from about 0.1% to about 1% by weight of a thickening agent; and
e) the balance an aqueous based carrier;
   for the use in making a medicament for treating mastitis in a mammal.

The term “effective amount” as used herein means “an amount of a composition as disclosed herein, effective at dosages and for periods of time necessary to achieve the desired or therapeutic result.” An effective amount may vary according to factors known in the art, such as the disease state, age, sex, species, and weight of the animal being treated. Although particular dosage regimes may be described in examples herein, a person skilled in the art would appreciated that the dosage regime may be altered to provide optimum therapeutic response. For example, several divided doses may be administered daily or the dose may be proportionally reduced as indicated by the exigencies of the therapeutic situation. In addition, the compositions of the present disclosure can be administered as frequently as necessary to achieve a therapeutic amount.

### EXAMPLES

### Process

The disclosed compositions can be prepared using the following procedurea:

### Example 1

Water is heated to approximately 50° C. in a holding tank. Begin stirring at 150 rpm with a suitable mixer, for example, a Cowls Mixer with a #5 blade (or equivalent). The biocide and preservative are then added and mixing continued until the solution is a homogeneous dispersion. The balance of the ingredients are then added in the following order: surfactant, emollient, penetrating agents. After the addition of each ingredient mixing is continued until the dispersion is homogeneous. At this point any fillers or inert materials can be added. Typically 2-3 minutes of mixing is necessary between the addition of each ingredient.

In a separate vessel, hydroxyethyl cellulose (in an amount approximately 0.05% wt. % of the final compositions) is pre-wetted using a ⅓ shaft vortex mixer. The hydroxyethyl cellulose solution is added over a period of 5-7 minutes. When the viscosity build is visible by a diminished or lack of vortex, the viscosity of the admixture is checked using a Brookfield RFV viscometer after the admixture is allowed to stand unstirred for about 45 minutes. The desired range is from about 15,000 to 20,000 using a #3 spindle at 500 rpm.

### Example 2

Propylene glycol (20 g) [available from UNIVAR™] and cetylpyridinium chloride (2.4 g) [available from VERTELLUS™] are combined in a beaker and mixed with a magnetic stirrer at a rate sufficient to create a vortex. Stirring is continued for 5-7 minutes or until the cetylpyridinium chloride dissolves. An admixture of ethoxylated caprylic & capric glycerol esters (5 g) available from ABITEC™ Corp. is added and the solution continuously stirred until homogenous.

Separately, urea (0.6 g) [available from UNIVAR™] is dissolved in water (50 mL). If necessary, the solution can be heated to about 70 ° C. to aid in the dissolution of the urea. The aqueous solution of urea is then added over approximately 0.5 minutes to the constantly stirred solution formed above. Once homogeneous, water (920.2 mL) is slowly added while mixing is continued at a rate that avoids the formation of any foaming. FDC Blue #1 available from SENSIENT™ Technologies is added in an amount equal to 0.001 wt. % of the final solution. If desired, a thickener, inter alia, Guar gum can be added. The final pH is from about 5.9 to about 6.8.

The following procedure can be used to evaluate the disclosed compositions against various microorganisms. The results below further indicate the effectiveness of the disclosed compositions as measured against state of the art iodine compositions.

### Comparative Test 1

A 1% solution of IODINE Teat Dip manufactured by AST Inc., Bernville, PA 19506 (control) is tested against a 0.1% of the composition disclosed in Example 1.

### Materials:

Eschericia coli—ATCC # 8739

Staphylococcus aureus—ATCC # 6538

Pseudomonas aeruginosa—ATCC # 9027

Nutrient agar plates (15 mm×100 mm) # DF0001-17—available from Fisher Scientific.

Incubator 35-37° C.—Precision Scientific Model #6.

A 0.1 mL sample of bacteria was pipetted onto an agar place and uniformly spread across the surface. The inoculum contained from about 1×10⁷ to 1×10⁸ cfu/mL. To one-half of the inoculated plates was charged 15 μL of the control solution and to the other one-half was charged 15 μL of the composition according to Example 1 from Table I. The plates were then incubated for 24 hours. The amount of inhibition is determined by measuring the size of the zones of inhibition in millimeters using digital calipers. Table A discloses the results of the example procedure described herein.

TABLE A

| Species | Control | Example 1 soln. |
|---|---|---|
| Eschericia coli | 14.22 | 19.18 |
| Staphylococcus aureus | 7.1 | 34.2 |
| Pseudomonas aeruginosa | 15.76 | 20.57 |

### Comparative Test 2

A 0.15% solution of IODINE Teat Dip manufactured by AST Inc., Bernville, Pa. 19506 (control) is tested against a 0.1% of the composition disclosed in Example 1.

### Materials:

Eschericia coli—ATCC # 8739

Staphylococcus aureus—ATCC # 6538

Pseudomonas aeruginosa—ATCC # 9027

Nutrient agar plates (15 mm×100 mm) # DF0001-17—available from Fisher Scientific.

Incubator 35-37—Precision Scientific Model #6.

A 0.1 mL sample of bacteria was pipetted onto an agar place and uniformly spread across the surface. The inoculum contained from about 1×10⁷ to 1×10⁸ cfu/mL. To one-half of the inoculated plates was charged 15 μL of the control solution and to the other one-half was charged 15 μL of the composition according to Example 1 from Table I. The plates were then incubated for 24 hours. The amount of inhibition is determined by measuring the size of the zones of inhibition in millimeters using digital calipers. Table B discloses the results of the example procedure described herein.

TABLE B

| Species | Control | Example 1 soln. |
|---|---|---|
| Eschericia coli | 13.12 | 16.71 |

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

1. A composition for improving teat and udder hygiene in a mammal, comprising:
a) from about 0.05% to about 8% by weight of a biocidal system, comprising:
i) at least about 50% by weight of a primary biocide; and
ii) at least about 5% by weight of a preservative component;
b) from about 0.05% to about 0.2% by weight of a surfactant;
c) from about 1% to about 3% by weight of an emollient system comprising:
i) at least about 20% by weight of an extradermal penetrating agent; and
ii) at least about 50% by weight of an emollient base;
d) from about 0.1% to about 1% by weight of a thickening agent; and
e) the balance an aqueous based carrier.

2. The composition according to claim 1, wherein the primary biocide is a quaternary ammonium salt comprising at least one aryl or heteroaryl unit.

3. The composition according to claim 1, wherein the primary biocide is chosen from (C₁₂-C₁₄ alkyl)(Cᵢ-C₂ dialkyl)benzyl ammonium salts, N-(C₁₂-C₁₈ alkyl)heteroaryl ammonium salts, and N-[(C₁₂-C₁₄ alkyl)(C₁-C₂ dialkyl)]heteroarylalkylene ammonium salts.

4. The composition according to claim 1, wherein the primary biocide is chosen from (C₁₂-C₁₄ alkyl)dimethylbenzyl ammonium chloride, (C₁₂-C₁₄ alkyl)dimethylbenzyl ammonium bromide, (C₁₂-C₁₄ alkyl)dimethylbenzyl ammonium hydrogen sulfate, cetyl pyridinium chloride, cetyl pyridinium bromide, and cetyl pyridinium hydrogen sulfide.

5. (canceled)

6. The composition according to claim 1, wherein the preservative component is chosen from urea, imidazolyl urea, hydantoin, dichlorodimethylhydantoin, bromochloro-dimethylhydantoin, dibromodimethylhydantoin, and biuret.

7. (canceled)

8. The composition according to claim 1, wherein the biocidal system comprises:
i) from about 75% to about 95% by weight of a primary biocide; and
ii) from about 5% to about 25% by weight of a preservative component.

9. The composition according to claim 1, wherein the biocidal system comprises:
i) from about 75% to about 95% by weight of cetyl pyridinium chloride; and
ii) from about 5% to about 25% by weight of urea.

10. The composition according to claim 1, wherein the surfactant is chosen from a linear alkyl ether sulfate, a linear alkyl sulfate, a polyoxyethylene C₆-C₁₂ alkylphenyl ether, polyoxyethylene sorbitan tri(C₁₂-C₁₈)-alkanoate, polyoxyethylene sorbitan di(C₁₂-C₁₈)-alkanoate, polyoxyethylene sorbitan mono(C₁₂-C₁₈)-alkanoate, or polyoxyethylene C₉-C₂₀ alkyl ether.

11. (canceled)

12. The composition according to claim 1, wherein the surfactant is chosen from sodium decyl sulfate, sodium dodecyl sulfate, sodium tetradecyl sulfate, potassium decyl sulfate, potassium dodecyl sulfate, potassium tetradecyl sulfate, ammonium decyl sulfate, ammonium dodecyl sulfate, ammonium tetradecyl sulfate, and mixtures thereof.

13. The composition according to claim 1, wherein the surfactant is a polyoxyethylene C₆-C₁₂ alkylphenyl ether having from about 8 to about 12 ethyleneoxy units.

14. The composition according to claim 1, wherein the nonionic surfactant is a polyoxyethylene(5) isooctylphenyl ether, polyoxyethylene(8) isooctylphenyl ether, polyoxyethylene(9) nonylphenyl ether, polyoxyethylene(10) isooctylphenyl ether, polyoxyethylene(branched) nonylphenyl ether, polyoxyethylene(12) nonylphenyl ether, polyoxyethylene(12) isooctylphenyl ether, polyoxyethylene(40) nonylphenyl ether, and polyoxyethylene(40) isooctylphenyl ether.

15. The composition according to claim 1, wherein the nonionic surfactant is polyethylene glycol 4-(1,1,3,3-tetramethylbutyl)-phenyl ether or a polyoxyethylene sorbitan mono-, di-, and tri-(C₁₂-C₁₈)-alkanoate.

16. (canceled)

17. The composition according to claim 1, wherein the nonionic surfactant is a polyoxyethylene(20) sorbitan trioleate, polyoxyethylene(20) sorbitan monooleate, polyoxyethylene(20) sorbitan monostearate, polyoxyethylene(20) sorbitan monopalmitate, and polyoxyethylene(20) sorbitan monolaurate or a polyoxyethylene C₉-C₂₀ alkyl ether.

18. (canceled)

19. The composition according to claim 1, wherein the nonionic surfactant is a polyoxyethylene C₉-C₂₀ alkyl ether chosen from C₉-C₁₁ alkyl-(5)-ethoxylate, C₉-C₁₁ alkyl-(6)-ethoxylate, C₉-C₁₁alkyl-(8)-ethoxylate, C₉-C₁₁ alkyl-(9)-ethoxylate, C₂-C₁₃ alkyl-(6.5)-ethoxylate, C₁₂-C₁₅ alkyl-(5)-ethoxylate, C₁₂-C₁₅ alkyl-(7)-ethoxylate, C₁₂-C₁₅ alkyl-(9)-ethoxylate, C₁₂-C₁₅ alkyl-(12)-ethoxylate, C₁₄-C₁₅ alkyl-(7)-ethoxylate, and C₁₁-C₁₅ alkyl-(7)-ethoxylate.

20-21. (canceled)

22. The composition according to claim 1, wherein the extradermal penetrating agent is a C₁-C₈ mono- or poly-hydroxy alcohol.

23. The composition according to claim 1, wherein the extradermal penetrating agent is chosen from benzyl alcohol, ethylene glycol, and propylene glycol.

24. (canceled)

25. The composition according to claim 1, wherein the emollient base is chosen from C₃-C₁₄ linear or branched alkyl alcohols, C₃-C₁₄ linear or branched polyols, C₆-C₁₄ di-esters of C₆-C₁₂ diacids, hydrocarbons, natural waxes, vegetable oils, and silicones.

26. The composition according to claim 1, wherein the emollient base is a polyol having the formula:
HOCH₂—[CHOH]ₓ—CH₂OH
wherein the index x is from 1 to 6.

27. The composition according to claim 1, wherein the emollient base comprises a polyol chosen from glycerol, (2R,3R)-butane-1,2,3,4-tetraol, (2S,3R)-butane-1,2,3,4-tetraol, (2R,35)-butane-1,2,3,4-tetraol, (2S,3S)-butane-1,2,3,4-tetraol, (2R,3R,4R)-pentane-1,2,3,4,5-pentaol, (2S,3R,4R)-pentane-1,2,3,4,5-pentaol, (2R,3S,4R)-pentane-1,2,3,4,5-pentaol, (2R,3R,4S)-pentane-1,2,3,4,5-pentaol, (2S,3S,4R)-pentane-1,2,3,4,5-pentaol, (2S,3R,4S)-pentane-1,2,3,4,5-pentaol, (2R,3S,4S)-pentane-1,2,3,4,5-pentaol, and (2S,3S,4S)-pentane-1,2,3,4,5-pentaol.

28. (canceled)

29. The composition according to claim 1, wherein the emollient base comprises glycerol and one or ethoxylated partial glyceride fatty acid esters.

30. The composition according to claim 1, wherein the emollient base further comprises branched chain esters, ethoxylated partial glyceride fatty acid esters, protein derivatives, lanolin, lanolin derivatives, fatty alcohol ethoxylates, emollient oils, fatty acids, fatty alcohols, and fatty alcohol esters.

31. The composition according to claim 1, wherein the emollient base further comprises an emollient base chosen from isononyl isonanoate, dioctyl sebacate, isooctyl isooctanoate, dioctyl adipate, squalane, petrolatum, mineral oil, carnauba wax, candelilla wax, beeswax, sunflower oil, sesame oil, olive oil, cyclomethicone, or dimethicone.

32. The composition according to claim 1, wherein the emollient' system comprises:
i) at least about 30% by weight of an extradermal penetrating agent; and
ii) at least about 60% by weight of an emollient base.

33. (canceled)

34. The composition according to claim 1, wherein the thickening agent is chosen from hydroxynethyl cellulose, hydroxyethyl cellulose, methylcellulose, hydroxypropyl cellulose, methyl cellulose, carboxy methylcellulose, emulsifying waxes, alkyl triammonium methosulfate, and ceteraryl octanoate.

35. The composition according to claim 1, wherein the thickening agent is chosen from hydroxynethyl cellulose and hydroxyethyl cellulose.

36. (canceled)

37. The composition according to claim 1, wherein the thickening agent is a natural gum.

38. The composition according to claim 1, wherein the thickening agent is chosen form Guar gum, Xanthan gum, Locust Bean Gum, Gum Arabic, and Carrageenan.

39. A composition according to claim 1, comprising:
a) from about 0.05% to about 3% by weight of a biocidal system, comprising:
i) at least about 75% by weight of a primary biocide; and
ii) at least about 5% by weight of a preservative component; and
b) from about 0.05% to about 0.2% by weight of a surfactant having an HLB of from about 10 to about 20.

40. A composition according to claim 1, comprising:
a) about 3% by weight of a biocidal system, comprising:
i) 90% by weight of cetyl pyridinium chloride; and
ii) 10% by weight of urea;
b) about 0.2% by weight of polyoxyethylene(10) isooctylphenyl ether;
c) about 3% by weight of an emollient system comprising:
i) about 33.3% by weight of propylene glycol; and
ii) about 66.7% by weight of glycerol;
d) about 0.5% by weight of hydroxyethylcellulose; and
e) the balance water.

41. A composition according to claim 1, comprising:
a) from about 0.05% to about 3% by weight of a biocidal system, comprising:
i) at least about 75% by weight of a primary biocide; and
ii) at least about 5% by weight of a preservative component;
b) from about 0.05% to about 0.2% by weight of a linear alkyl sulfate;
c) from about 1% to about 3% by weight of an emollient system comprising:
i) at least about 20% by weight of an extradermal penetrating agent; and
ii) at least about 50% by weight of an emollient base;
d) from about 0.1% to about 1% by weight of a thickening agent chosen from Guar gum, Xanthan gum, Locust Bean Gum, Gum Arabic, and Carrageenan; and
e) the balance an aqueous based carrier.

42. (canceled)

43. A method for treating or preventing mastitis in an infected or exposed mammal comprising, contacting an effective amount of a biocidal composition to the mammal, a composition comprising:
a) from about 0.05% to about 3% by weight of a biocidal system, comprising:
i) from about 75% to about 95% by weight of a primary biocide; and
ii) from about 5% to about 25% by weight of a preservative component;
b) from about 0.05% to about 0.2% by weight of a nonionic surfactant having an HLB of from about 13 to about 16;
c) from about 1% to about 3% by weight of an emollient system comprising:
i) at least about 30% by weight of an extradermal penetrating agent; and
ii) at least about 60% by weight of a emollient base;
d) from about 0.25% to about 0.75% by weight of a thickening agent; and
e) the balance an aqueous based carrier.

44. A method according to claim 43, comprising, contacting an effective amount of a biocidal composition to the mammal, a composition comprising:
a) about 3% by weight of a biocidal system, comprising:
i) 90% by weight of cetyl ammonium chloride; and
ii) 10% by weight of urea;
b) about 0.2% by weight of polyoxyethylene(10) isooctylphenyl ether;
c) about 3% by weight of an emollient system comprising:
i) about 33.3% by weight of propylene glycol; and
ii) about 66.7% by weight of glycerol;
d) about 0.5% by weight of hydroxyethylcellulose; and
e) the balance water.

45. A method for treating or preventing mastitis in an infected or exposed mammal comprising, applying an effective amount of a biocidal composition to the mammal, a composition comprising:
a) from about 0.05% to about 8% by weight of a biocidal system, comprising:
i) at least about 75% by weight of a primary biocide; and
ii) at least about 5% by weight of a preservative component;
b) from about 0.05% to about 0.2% by weight of a nonionic surfactant having an HLB of from about 10 to about 20;
c) from about 1% to about 3% by weight of an emollient system comprising:
i) at least about 20% by weight of an extradermal penetrating agent; and
ii) at least about 50% by weight of an emollient base;
d) from about 0.1% to about 1% by weight of a thickening agent; and
e) the balance an aqueous based carrier.

46-51. (canceled)
